SEQUENCE LISTING (6 sequences)

SEQ ID NO: 1 - DNA, 500 bp, Homo Sapiens
gagccagctcccgcactgctaggatcctgttggcttcctctggcacggctcggctgggttcctccctccctgtgcggctcgcctcagcaggcacacatttagaaatcattcacgagcccctcaaagtcgcacaaaagaactgcatgggaaagtaggaagagctgtctgcaccaagggactcctggtttccacgggaatggagtagctctctgactgtctcgttcatttcatcagacctccctctatgtgtatgtcataagctgcaaggtagcaacagccaggagggcggaccaaacaggctttttcttctccctctttttgctacatattaatattgggaagttttcctttgcttttgagagaaactggagaaatggccttttgtgcagattcccattaaggtaggtaagtggcactgtggtaattttttaggctgaagggtgaagagagaacataaataaggctagaaaacagtatgtcctcggagtgctgtgagtgtc

SEQ ID NO: 2 - DNA, 500 bp, Homo Sapiens
ggcacttccatccaaagccaacagtgtttgtgtccagagtggaattactgacattggccacataggctcagggtggctaggcacgtctgtggtgataactctgataaactattagcactatttttatttaatagatacaccattgaactggcttattttcttcagcagaaatatgccacccagatattattcaaaacctcacatgtggtaggaaataagttggtttcgcagtaccaatttttttcccccaccagtaatgacaacttgccttacttgtaaagaaagccctttcccaagtaggtttctaaaggaggcagttcgatctctctctttttgcaggcatgaaaatattttcctcaatagttgggttttgctacagttctatcaccttctgttcttcacattctccctggacaaattcaaccactctcatgccttcaatatgtttgtggccaagtctgtaccttcatagctgattatttctctcagttccagaccta

SEQ ID NO: 3 - DNA, 15 bp, Artificial Sequence
  Modified DNA Sequence
ctgtgagtgtctggc

SEQ ID NO: 4 - DNA, 15 bp, Artificial Sequence
  Modified DNA sequence
ctgtgagtgtccggc

SEQ ID NO: 5 - DNA, 32 bp, Artificial Sequence
  Modified DNA sequence
agagagaacataaataaggctagaaaacagta

SEQ ID NO: 6 - DNA, 20 bp, Artificial Sequence
  Modified DNA sequence
cactgttggctttggatgga

(19) United States
(12) Patent Application Publication
(10) Pub. No.: US 2014/0162261 A1
(43) Pub. Date: Jun. 12, 2014

(54) DETECTION KIT FOR IDENTIFYING GENOTYPE IN DEPRESSION PATIENTS AND METHOD OF USING THE SAME

(71) Applicants: Wen E., Beijing (CN); Qingmei Kong, Beijing (CN); Xiaojing Zhang, Beijing (CN); Hong Shao, Beijing (CN); Zhenguo Zhao, Beijing (CN); Shuping Liu, Beijing (CN); Maomao Li, Beijing (CN); Xinxia Tian, Beijing (CN)

(72) Inventors: Wen E., Beijing (CN); Qingmei Kong, Beijing (CN); Xiaojing Zhang, Beijing (CN); Hong Shao, Beijing (CN); Zhenguo Zhao, Beijing (CN); Shuping Liu, Beijing (CN); Maomao Li, Beijing (CN); Xinxia Tian, Beijing (CN)

(21) Appl. No.: 14/091,575

(22) Filed: Nov. 27, 2013

(30) Foreign Application Priority Data
Dec. 9, 2012 (CN) 201210523154

(51) Int. Cl. C12Q 1/68

(52) U.S. Cl. CPC C12Q 1/6883
USPC 435/6.11

(57) ABSTRACT

The present invention relates to a rs6311 test kit, which includes a probe, a primer, and a polymerase chain reaction solution, wherein said probe sequence is as follows: rs6311T-fam: CTGTGAGTGTCTGGC (SEQ. ID. NO. 1) and rs6311C-vic: CTGTGAGTGTCCGGC (SEQ. ID. NO. 2); and said primer sequence is as follows: rs6311-F: AGAGAGAACATAAATAAGGCTAGAAAACAGTA (SEQ. ID. NO. 3) and rs6311-R: CACTGTTGGCTTTGGATGGA (SEQ. ID. NO. 4). The test kit is used to determine genotype of a depression patient, in order to treat the depression patient with a combination of serotonin reuptake inhibitors (SSRI) and low dose risperidone. The actual dose of risperidone and the ratio between SSRIs and risperidone is determined by the genotype of the depression patient. The present invention determines human drug metabolism rate through a single nucleotide polymorphism and provides a platform to adjust the patient's treatment.

### CROSS REFERENCE TO RELATED APPLICATION

This application claims priority to a Chinese Patent application, No. CN 201210523154, filed on Dec. 9, 2012, which is incorporated herein by reference.

### TECHNICAL FIELD OF THE INVENTION

The invention relates to the determination of genotype in a depression patient, particularly relates to an rs6311 test kit, detection method and its application.

### BACKGROUND OF THE INVENTION

A common problem in treating depression patients is that an antidepression drug may be very effective on some patients, but much less effective or completely ineffective on others due to the reason of their genome differences. There are many manifestations of human genome on a single base mutation, i.e., a single nucleotide polymorphism (SNP). Single nucleotide polymorphism on the genome is a single nucleotide mutation on the formation of genetic markers. There are many forms of polymorphisms. Therefore, SNP became the third generation of genetic markers. Many human phenotypic differences, drug or disease susceptibility, etc., may be associated with SNP.

Depression is currently the world's fourth largest disease. By 2020, it could become the second largest disease after heart disease and is becoming a serious global problem. Studies suggest that depression may be related to low content of central noradrenaline (NA), 5-hydroxytryptamine (5-HT), dopamine (DA), monoamine oxidase (MAO) and other chemicals or a receptor dysfunction. Since 1950's, development in antidepressant drugs has grown rapidly. Clinical treatment of depression has also made great progress. Stahl divided antidepressants into seven categories. Among them, serotonin reuptake inhibitors (SSRI) are the most widely used first-line antidepressant drugs in Europe and the United States to treat depression. (Stahl, 1998)

In 1988, Eli Lilly Company introduced the first selective SSRI—fluoxetine. The mechanism of SSRIs is mainly through selective inhibition of presynaptic neurons serotonin (5-HT) pump in 5-HT reuptake, thereby increasing the synaptic concentration of 5-HT, to enhance 5-HT system function and achieve an antidepressant effect. SSRIs almost does not affect other nerve receptors (such as histamine receptors, acetylcholine receptors, adrenergic receptors, fast sodium channel, NE reuptake pumps, etc.), so action sites of SSRIs, compared to other conventional antidepressants, make them markedly faster, with smaller dosage, and significantly lower side effects (Kessler 2003. Nememff 2004, Finfgeld 2004, Masand 2002). Currently, SSRIs have reached more than 30 kinds, including fluoxetine, fluvoxamine, paroxetine (Paxil), sertraline (Zoloft), zimeldine, citalopram, and trazodone. Although there is no common chemical structure, they have common pharmacological characteristics: inhibit neuronal reuptake of 5-HT, while having almost no significant impact on the other neurotransmitters.

SSRIs Commonly Used Clinical Dosage

Sertraline treatment of depression: 1 time a day, 50 mg/time. The therapeutic dose range is 50 mg˜100 mg for a day.

Daily doses of fluoxetine hydrochloride is 20 mg;

Paroxetine daily dose is 20 mg, taken once in the morning; adjustment in 10 mg increments 2 to 3 weeks after initial dose according to the disease; maximum daily dose is 50 mg. Elderly patients should not exceed the maximum dose of 40 mg daily. If taken for long term, a gradual reduction is needed with no abrupt stop.

Fluvoxamine daily dose is 100˜200 mg; 1 to 2 times daily with meals or after meals; dose adjustment should not exceed 300 mg daily.

Citalopram, initial dose 20 mg for adults; may be increased to 40 mg; if necessary, may be increased to 60 mg; Halved in patients below 65 years of age;

Escitalopram, initial dose is 10 mg daily; may increase to 20 mg daily after a week; orally in the morning or in the evening. Under normal circumstances a full therapy should take months or even longer. Elderly patients or hepatic dysfunction once 10 mg daily; no need for dose adjustment for mild or moderate renal insufficiency patients. Severe renal insufficiency should take caution.

Although SSRIs made great progress in the clinical treatment of depression, the long-term efficacy in a large number of patients showed that there is a big difference using SSRIs alone in the clinical treatment of depression patients. For patients that SSRIs treatment effect was not obvious, clinically they were treated with low dose (0.5-1.5 ing/day) risperidone, combined with SSRIs treatment for a short term (1-2 days). Most patients treated with this method showed a very good therapeutic effect (O'Connor and Silver. 1998; Ostroff and Nelson. 1999). Adding risperidone can improve the efficacy of antidepressant SSRIs. But this remarkable efficacy is not shown in all patients (Gerard J Marek, 2003). Researchers conducted genomics study and suggested that this phenomenon is associated closely with the patient gene polymorphisms, such as cytochrome P450 enzyme polymorphism, serotonin receptor gene polymorphism and so on.

5-HT_{2A} receptor gene is located on chromosome 13q 14.21. It has three exons and two introns. Turechi and other researchers suggested that brain 5-HT_{2A} receptor density is correlated with 5-HT_{2A} receptor gene polymorphism. Currently found in a variety of polymorphism in the gene, 1438G/A (rs6311C/T) polymorphism is the only 5-HT_{2A} receptor in the promoter region of the structural gene polymorphic variation, affecting the promoter activity. The promoter is a key transcription site. The difference in its sequence may result in different transcriptional changes, affecting the number of receptors, conformation, and binding function, leading to the central nervous system neurotransmitters and receptors changes and presynaptic changes, such as neurotransmitter synthesis, release, metabolism, and (or) re-uptake, and postsynaptic changes, such as receptors, conversion agent (G-protein), second messenger (phosphatidylinositol cyclase system) changes and ion transport abnormalities, which in turn will affect the efficacy of SSRIs.

In recent years, there have been many studies on rs6311 polymorphism and mood disorders, but the results lack of consistency. In a study in Asia, it was found that 5-HT_{2A} receptor gene 1438G/A polymorphism is correlated with paroxetine and fluoxetine treatment response. The treatment has better effects on 1438G/G genotype patients. In a study in Korea, 1438G/G genotype patients showed better response to citalopram. Moreover, this result was validated in a study in the Chinese Han population. In 2009, it was reported the 5-HT_{2A}rs6311 frequency increases at C allele and decreases at T allele in neurological disorders in Han population, and the frequency increase in patients with depression are prevalent. There are a lot of data to suggest 5-HT_{2A} receptor gene rs6311 polymorphism is associated with SSRIs efficacy and it is speculated that T allele, TT genotype may be a predictor of poor efficacy. In a Beijing Han population survey, T allele frequency is 48.8%; TT and TC genotypes were 69.8% among the population. (See rs6311 polymorphism population distribution in FIG. 1)

A study found that, SSRIs reduced 5-HT_{2A} receptor expression (Yatham et al, 1999). Meanwhile, other studies indicated that long-term use of blocking presynaptic membrane on 5-HT reuptake effects of SSRIs can cause a decrease in cortical 5-HT_{2A} receptor density. Glennon and Dukat reported SSRIs is correlated with a reduction in 5-HT_{2A} receptor reaction rate (Glennon et al, 1995). Accordingly, it is hypothesized that locus in rs6311 T allele. TT genotype may reduce 5-HT_{2A} receptor expression or its reactivity with SSRIs. The use of specific 5-HT_{2A} receptor antagonist agent may improve the treatment of depression.

SSRIs in combination with low-dose risperidone (0.5-1 mg/day) can significantly improve most of the patient's symptoms in 1-2 days. (O'Connor and Silver, 1998; Ostroff and Nelson, 1999). Risperidone may be completely digested after oral administration, reach peak plasma concentration within 1-2 hours, and its digestion is not affected by food. Risperidone is a monoaminergic antagonist with unique selectivity properties, which has high affinity with 5-HT2 receptors. Risperidone binding capacity with 5-HT_{2A} receptor is far greater than with 5-HT_{1A} and 5-HT_{2C} receptors, which is about 1,000 times higher than binding capacity with 5-HT_{1A} receptor. It can also bind with the adrenergic receptor and the low affinity H1-histamine receptors and α2-adrenergic receptors. Risperidone does not bind with cholinergic receptors. Effective dose of risperidone in treatment of depression is 0.5-1 mg/day. The dose for the treatment of schizophrenia was 6 mg/day. This depends on the different receptors at different concentrations that risperidone selectively hinders. Risperidone of 4 mg dose blocks 70-80% striatal dopamine D2 receptors (Nyberg et al, 1999). Further increase in plasma concentration may cause increase in extrapyramidal side effects. Risperidone of 0.5-1 mg dose may saturate and close 5-HT_{2A} receptor, and reduce the extrapyramidal side effects to a minimum.

5-HT_{2A} receptors are the main targets for SSRIs and risperidone, which belong to the G protein-coupled receptor family, mainly in the frontal cortex (Arora R C et al. 1989; Yates M et al. 1990). 5-HT_{2A} receptor can activate non-5-HT_{2A} receptor during treatment of antagonist depression and other neuropsychiatric diseases (Gerard J Marek, 2003). Researchers in experimental animal models of depression found that 5-HT_{2A} may regulate the body's response to drugs. Black and Goodwin's study found that antidepressant treatment can reduce 5-HT_{2A} receptor density in animal brains (Goodwin G M et al. 1985). Biegon et al reported that 5-HT_{2A} receptor binding capacity on platelet membranes in patients with depression is correlated with the patients' clinical symptoms. When the patients showed clinical improvement, the 5-HT_{2A} receptor binding capacity decreased significantly when patients' clinical symptoms do not improve, 5-HT_{2A} receptor binding capacity does not change (Biegon A Essar N et al. 1990). These findings strongly suggest there is a close correlation between 5-HT_{2A} receptor and antidepressant drug response. Low dose risperidone can selectively block 5-HT_{2A} receptor. Given that in the treatment of depression of 5-HT system plays a very complex role, a variety of 5-HT receptor activation in the treatment will affect SSRIs efficacy. 5-HT_{2A} receptor-specific closure may have brought new ways for the treatment of depression. (Ostroff and Nelson, 1999: Ansoms et al, 1977).

In order to accurately use SSRIs, the present invention designs and synthesizes specific probes and primers to detect human genome rs6311 loci polymorphism (See rs6311 polymorphism genome sequences in FIG. 2). The patients are divided into normal metabolic group (CC genotype) and slow metabolic group (CT and TT genotype). Normal metabolic group is routinely administration SSRIs. For CT and TT groups, with the combination of SSRIs drugs, there is a simultaneous administration of low doses of risperidone (0.5-1 mg/day), to obtain a good therapeutic effect, while greatly reduce the side effects of the drugs.

### SUMMARY OF THE INVENTION

The present invention comprises of an individualized detection kit, which contains a primer, a probe, and other reagents. The probe is specifically design for the detection of human metabolism genotypes. The components in the detection kit can be used in combination of extracted DNA from a depression patient to carry out polymerase chain amplification reaction such that the genotypes can be determined.

The present invention comprises of the following detection method.

Selectively enroll patients based on evaluation criteria, according to research method flow diagram (FIG. 3), in accordance with the technical plan (FIG. 4). Take 2 ml peripheral blood (EDTA anticoagulant) from a patient. Extract genomic DNA from whole blood. Bi-directional sequencing methods and real-time fluorescence analysis, such as Taqman probe techniques, are used to determine the genotype of the patient. Patients are then randomly divided into two groups; one group is administered drugs by conventional means and the effect is evaluated. Another group undergoes Set A treatments: a group with CC genotype is administered in accordance with common clinical practice; genotype CT/TT groups are given clinical dose of SSRIs, combined with low dose risperidone. According to the clinical evaluation of effects of this group, Set B treatment can be further carried out: CC genotype is administered in accordance with conventional regimens. CT/TT genotypes are administered in accordance with reduced dose of SSRIs. Severity of symptoms of depression is evaluated. Initial dosing regimen can be derived based on the above experimental results.

Re-evaluation after the treatments to determine whether to exclude a patient or continue the treatment. The proportional relationship of ketanserin in combination with SSRIs for treatments of mild, moderate, and severe depression is further determined.

According to the above treatment plan, the present invention can achieve the following objectives:
To provide a detection kit for the classification of depression patients.
To adjust the ratio of risperidone and SSRIs in patients with depression medication.
Use two drugs that have different targets in order to improve the treatment.

### BRIEF DESCRIPTION OF DRAWINGS

FIG. 1 is an rs6311 polymorphism population distribution.

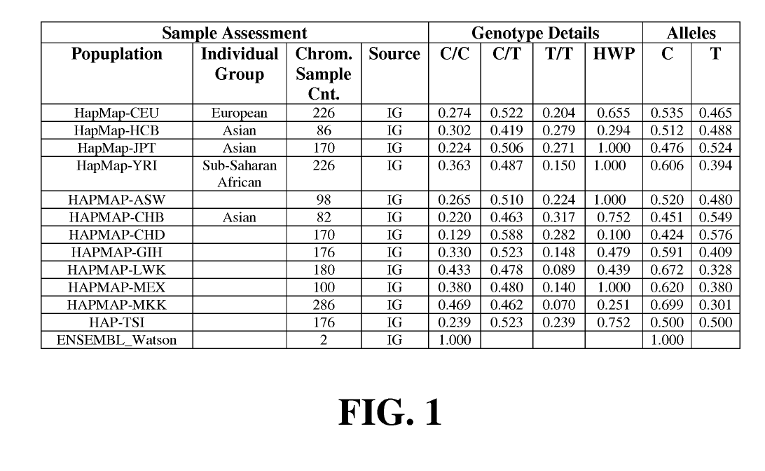

FIG. 2 is an rs6311 polymorphism genome sequence.

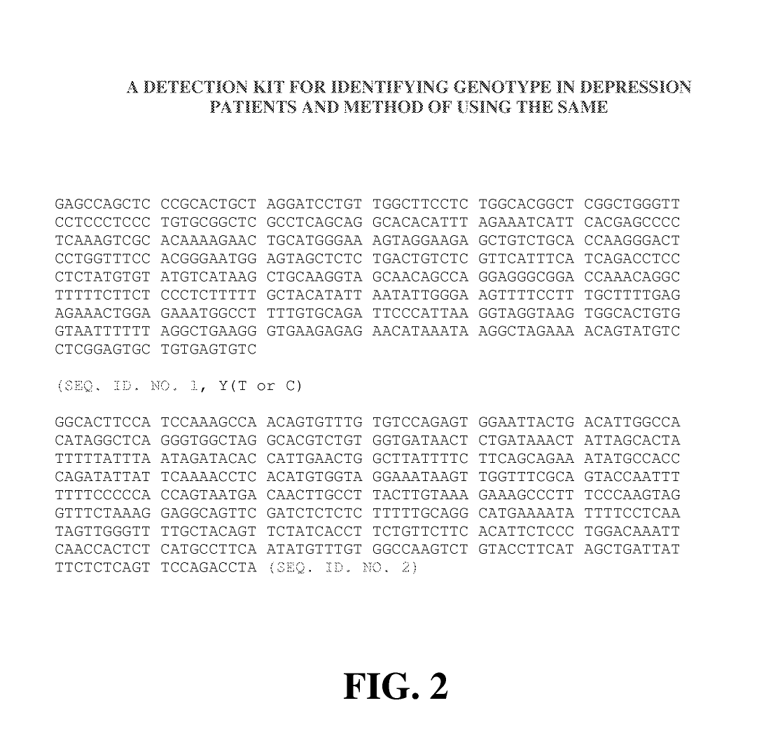

FIG. 3 is the method to classify patients.

FIG. 4 is technical plan for patient treatment.

### DETAILED DESCRIPTION OF THE INVENTION

The present invention comprises of a detection method and a detection kit to more easily use the detection reagent in the kit for the detection of the normal metabolism of population group (CC genotype) and slow metabolism (CT- and TT genotypes) populations in general population. One example of the detection method in the present invention, comprises the steps of:
1. Whole blood genomic DNA extraction. The extraction method is as follows: 10 microliters of proteinase K and 100 microliters of sample to be tested (EDTA anti-coagulated whole blood) are added in a 1.5 milliliter centrifuge tube; mix; centrifuge at 2000 rpm for 10 seconds; add 200 microliters of buffer solution B; mix by inversion; set at 56° C. for 10 minutes, during which the solution is mixed by inversion 2-3 times; add 200 microliters of anhydrous ethanol; mix by inversion; pour the solution into an adsorption column; centrifuge at 12000 rpm for 30 seconds; drained residual solution; place the adsorption column back into a collection tube; add to the adsorption column 500 microliters buffer C; centrifuge at 12000 rpm for 30 seconds; discard residual solution; place the adsorption column back into the collection tube; add 700 microliters of rinse solution W2; centrifuge at 12000 rpm for 30 seconds; discard residual solution; place the adsorption column back into the collection tube; add 500 microliters of rinse solution W2 to the adsorption column; centrifuge at 12000 rpm for 30 seconds; discard residual solution; place the absorption column back into the collection tube; and then centrifuged at 12000 rpm for 2 minutes; place the adsorption column into a new 1.5 microliter centrifuge tube; set at room temperature for a few minutes to completely dry the rinse solution from the residual adsorbent material; add to the middle of the adsorption film drop wise 100 microliters of elution buffer TE; set at room temperature for 2-5 minutes; centrifuge at 12000 rpm for 2 minutes to collect genomic DNA in the tube. Proteinase K, buffer solutions B, C and TE, and rinse solution W2 are commercially available. They are specified for illustration purposes only and are not inclusive.
2. PCR amplification, as follows:

Independently design primers and probes from the following sequence:

(SEQ. ID. NO. 1, Y = T or C)
GAGCCAGCTC CCGCACTGCT AGGATCCTGT TGGCTTCCTC
TGGCACGGCT CGGCTGGGTT CCTCCCTCCC TGTGCGGCTC
GCCTCAGCAG GCACACATTT AGAAATCATT CACGAGCCCC
TCAAAGTCGC ACAAAAGAAC TGCATGGGAA AGTAGGAAGA
GCTGTCTGCA CCAAGGGACT CCTGGTTTCC ACGGGAATGG
AGTAGCTCTC TGACTGTCTC GTTCATTTCA TCAGACCTCC
CTCTATGTGT ATGTCATAAG CTGCAAGGTA GCAACAGCCA
GGAGGGCGGA CCAAACAGGC TTTTTCTTCT CCCTCTTTTT
GCTACATATT AATATTGGGA AGTTTTCCTT TGCTTTTGAG
AGAAACTGGA GAAATGGCCT TTTGTGCAGA TTCCCATTAA
GGTAGGTAAG TGGCACTGTG GTAATTTTTT AGGCTGAAGG
GTGAAGAGAG AACATAAATA AGGCTAGAAA ACAGTATGTC
CTCGGAGTGC TGTGAGTGTC
(SEQ. ID. NO. 2)
GGCACTTCCA TCCAAAGCCA ACAGTGTTTG TGTCCAGAGT
GGAATTACTG ACATTGGCCA CATAGGCTCA GGGTGGCTAG
GCACGTCTGT GGTGATAACT CTGATAAACT ATTAGCACTA
TTTTTATTTA ATAGATACAC CATTGAACTG GCTTATTTTC
TTCAGCAGAA ATATGCCACC CAGATATTAT TCAAAACCTC
ACATGTGGTA GGAAATAAGT TGGTTTCGCA GTACCAATTT
TTTTCCCCCA CCAGTAATGA CAACTTGCCT TACTTGTAAA
GAAAGCCCTT TCCCAAGTAG GTTTCTAAAG GAGGCAGTTC
GATCTCTCTC TTTTTGCAGG CATGAAAATA TTTTCCTCAA
TAGTTGGGTT TTGCTACAGT TCTATCACCT TCTGTTCTTC
ACATTCTCCC TGGACAAATT CAACCACTCT CATGCCTTCA
ATATGTTTGT GGCCAAGTCT GTACCTTCAT AGCTGATTAT
TTCTCTCAGT TCCAGACCTA

MIX is commercially available, for example, GeneCopoeia company 2* Probe AllinOne Q-PCR Mix. Add MIX, probe, primer, and the extracted genomic DNA at a pre-determined ratio, and add paraffin oil; use a fluorescent polymerase chain reaction (PCR) instrument in accordance with the following procedure for amplification: 95° C. 10 minutes—40 cycles (95° C. 10 seconds −60° C. 30 seconds).
3. Determine genotype according to the amplification curves for single chain polymorphism genotype sample, as follows:
Use a single strand polymorphism analyzer, such as BIO-RAD iQ5 instrument; detect the genotype by a common detection method, such as Taqman fluorescent PCR detection method. Different genotypes show different results. Normal metabolism group (CC genotype) shows the results as: VIC labeled probe amplification; slow metabolism groups (CT- and TT genotypes) show the results as: FAM-labeled probe and VIC-labeled probe amplified simultaneously, or FAM-labeled probe amplification.
Administer medicine according to the genotype classifications. For example, for the normal metabolism of group (CC genotype), use general administration of SSRIs.
For slow metabolism groups (CT- and TT genotypes): administer SSRIs, with simultaneous administration of low doses of risperidone (0.5-1 mg/day); evaluate efficacy; and adjust the dosage accordingly. In the above method, the whole blood genomic DNA extraction can be done using commercially available DNA extraction kits, for example, Blood Genomic DNA Miniprep Kit manufactured by Beijing Zoman Biotechnology Co., Ltd. The kit contains erythrocyte lysis buffer, buffer A, B, C, rinse solution W2, elution buffer TE, proteinase K, adsorption columns, collection tubes, brochures, etc. The kit can be obtained commercially. Blood genomic DNA extraction can be carried out according to the prior art methods to extract and is not limited to the above-mentioned method.

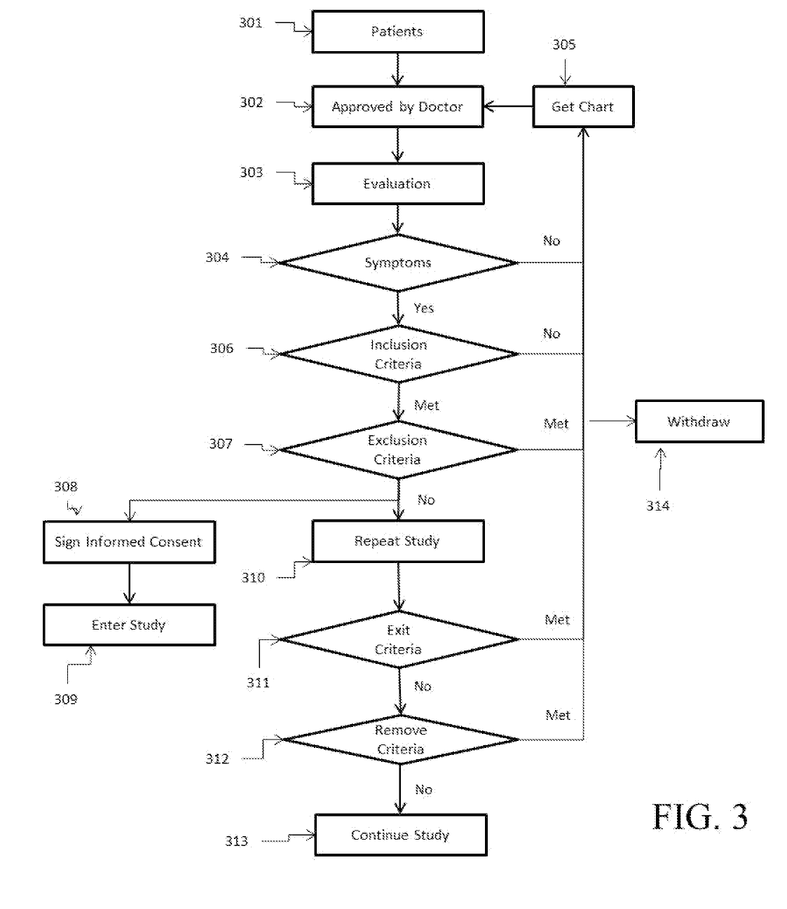

FIG. 3 shows the research method for the detection method and detection kit. Patients (301) must be evaluated by their doctors before entering the study (302). The patients are evaluated (303) and their symptoms are recorded (304) to determine whether they are suitable for the study. If there is no symptom of depression, the patients are sent back to their doctors (305). If the patients' symptoms are qualified for the study, then they are evaluated based on inclusion criteria (306). If the inclusion criteria are not met, the patients are sent back to their doctors (305). If the inclusion criteria are met, the patients are evaluated based on exclusion criteria (307). If the exclusion criteria are met, the patients are sent back to their doctors (305). If the exclusion criteria are not met, the patients can sign an informed consent (308) and enter the study (309). Or, the patients enter repeat study (310). During the repeat study, the patients will be evaluated based on exit criteria (311). If the exit criteria are met, the patients finish the study and are sent back to their doctors (305). If the exit criteria are not met, the patients are evaluated further based on a remove criteria (312). If the remove criteria are met, the patients are removed from the study and sent back to their doctors (305). If the remove criteria are not met, the study is continued (313). At any stage of the study, a patient may withdraw voluntarily (314) and the study will be terminated on that patient.

Evaluation Criteria

| | |
|---|---|
| Operational | (1) Where the treatment group patients attending doctors evaluate whether |
| Process Flow | patients pass an initial test. |
| | (2) Evaluate the patients' condition, inclusion criteria and exclusion criteria |
| | one by one to determine whether the patients fit in the study. |
| | (3) If the patients fit in the study then the patients signed informed consent |
| | and be assigned a serial number. |
| | (4) Fill the patients' condition in a detailed document. |
| | (5) Re-evaluation after the study to determine whether to exclude a patient |
| | or continue to study |
| | (6) Follow-up with the patients. |
| Inclusion Criteria | (1) Age 5-60 years old. |
| | (2) Electrocardiogram and myocardial enzymes are normal (but isolated |
| | asymptomatic T wave flat is not exclusion criteria). |
| | (3) ALT and AST in liver function tests less than 1.5 times the upper limit |
| | of normal value; BIL normal without underlying liver disease. |
| | (4) Urine and renal function indicators are within the normal range. |
| | (5) Meet ethical requirements; patients voluntarily signed informed consent. |
| Exclusion Criteria | (1) Has other adverse drug addiction and/or long-term alcoholics. |
| | (2) Active infection or other serious underlying disease. |
| | (3) Had clinically significant heart disease or myocardial infarction in past |
| | 12 months and have currently uncontrolled hypertension. |
| | (4) Patients with uncontrolled seizures, central nervous system disorders, |
| | and cannot sign informed consent or with bad reactions observed or |
| | mentally incapacitated. |
| | (5) Have previous history of other malignancies. |
| | (6) Prior to the study involved any experimental drug studies, including |
| | traditional Chinese medicine, qigong and other homeopathy; |
| Exit Criteria | (1) Patients require to withdraw. |
| | (2) Mishaps in the study. |
| | (3) During the study, it is determined the study should not continue and/or |
| | patients unable to continue this study. |
| | (4) Simultaneous use of other drugs affecting the efficacy of evaluation. |
| | (5) Clinical data are incomplete. |
| Remove criteria | No clinical data recorded; or targets too small to record final data processing |
| | and statistics. |

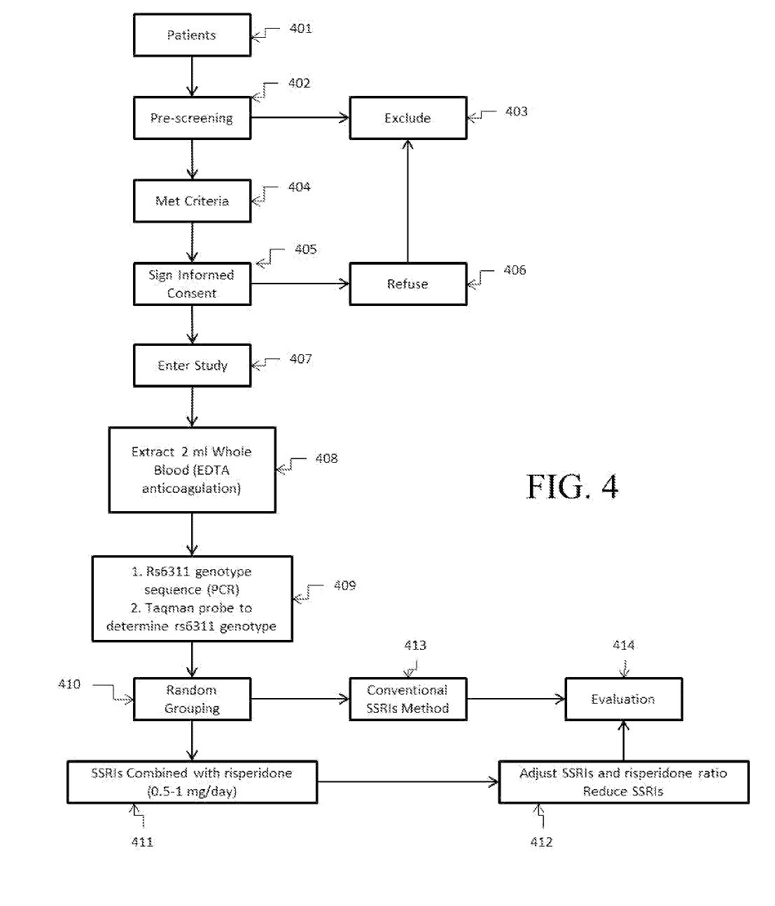

FIG. 4 is the technical plan for the study. Patients (401) are prescreened (402) according the inclusion and exclusion criteria as well as their symptoms. If the patients are determined not meeting the criteria, they are excluded from the study (403). If the criteria are met (404), the patients will be asked to sign an informed consent (405) and enter the study (407). If a patient refuses to sign the informed consent (406), he or she will be excluded from the study (403). A 2 milliliter whole blood sample (EDTA anti-coagulated) is extracted from a patient (408). The genomic DNA will be extracted and undergo an rs6311 genotype sequencing and amplification through a polymerase chain reaction (PCR); and an rs6311 genotype will be determined by common methods, for example, Taqman probe method (409). The patients are then divided into study groups according to their genotypes (410). For fast metabolism group (CC genotype), conventional SSRIs administration is performed (413). For slow metabolism groups (CT and TT genotypes), SSRIs are administered in combination with a low dose risperidone (0.5-1 mg/day) (411). The ratio of SSRIs and risperidone is adjusted based on evaluation and the usage of SSRIs is reduced if necessary (412). Long term efficacy of the SSRIs are evaluated (414).

According to the above detection method, the present invention also comprises a detection kit. The kit includes a probe, a primer and MIX reaction solution (conventional PCR reaction solution, commercially available).

Wherein said probe sequence is as follows:

(SEQ. ID. NO. 3)
rs6311T-fam CTGTGAGTGTCTGGC
(SEQ. ID. NO. 4)
rs6311C-vic CTGTGAGTGTCCGCTC

Wherein said primer sequence is as follows:

(SEQ. ID. NO. 5)
rs6311-F AGAGAGAACATAAATAAGGCTAGAAAACAGTA
(SEQ. ID. NO. 6)
rs6311-R CACTGTTGGCTTTGGATGGA

The probe and primer described above can be synthesized according to the art, such as the use of synthetic devices, or by chemical synthesis, through nucleotide connection. For example, probe synthesis can utilize the MGB probe marking method and purity of the product should be above 99%.

The test kit contains these reagents according to the proportion they are costumed, wherein the dosages of the probe and the primer can be 1-3× of the actual usage, according to the sensitivity of the instrument. The reagents may be individually packaged, then packaged together in the same box. The test kit must be placed in cold storage. An operational manual is also included. The reagents may be packaged according to needs and may be packaged with whole blood genomic DNA extraction kit so that it is easy to use them together.

Advantages of the present invention include:
(1) It determines human drug metabolism rate through a single nucleotide polymorphism and provides a platform to adjust the patient's treatment plan. It also provides a theoretical and practical base for the application of single nucleotide polymorphisms in clinical treatment.
(2) It provides new ideas for treating depression as well as new treatment options to improve the quality of life for patients.
(3) It provides a new treatment plan for patients with drug resistance to SSRIs.
(4) It reduces the amount of drugs without affecting the efficacy of SSRIs and minimizes adverse effects of SSRIs to depression patients.

### Specific Embodiments

### Best Mode

### Fluorescence Amplification Kit and its Application

The kit contains: a fluorescent reaction tube, triple-distilled deionized water, MIX reaction solution, wherein the fluorescent reaction tube contains the following primers and probes:

said probe sequence is as follows:

(SEQ. ID. NO. 3)
rs6311T-fam CTGTGAGTGTCTGGC
(SEQ. ID. NO. 4)
rs6311C-vic CTGTGAGTGTCCGGC

said primer sequence is as follows:

(SEQ. ID. NO. 5)
rs6311-F AGAGAGAACATAAATAAGGCTAGAAAACAGTA
(SEQ. ID. NO. 6)
rs6311-R CACTGTTGGCTTTGGATGGA

and paraffin oil.

The reagents are mix-packed and stored at −20° C.

The reagent preparation method is as follows:
(1) Probe preparation: 25 micro-molar, triple-distilled deionized water as the solvent.
(2) Primer preparation: 20 micro-molar, triple-distilled deionized water as the solvent.
(3) The ratio of reagents is as follows:
   a. 0.1 microliter of each probe:
   b. 0.8 microliter of each primer;
   c. Triple-distilled deionized water in 1.5 milliliter vial;
   d. MIX reaction solution in 1.5 milliliter vial:
   e. Paraffin oil may be added directly to the fluorescent reaction tube. It may also be individually packaged and mix with other reagents during reaction.
(4) Apply a layer of paraffin oil on top of the mixture during the PCR amplification to reduce the variability due to evaporation of the liquid during the PCR process. Studies have shown that application of paraffin oil in increase amplification production by five-fold.
(5) Use of the kit as follows:
   a. DNA extraction according to the following steps:
      1) Whole blood genomic DNA extracted using the genomic DNA of blood Miniprep Kit (Blood Genomic DNA Kit). The kit contains erythrocyte lysis buffer, a buffer A, B, C, rinse solution W2, elution buffer TE, proteinase K, adsorption columns, collection tubes, manuals, etc.
      2) Instrumentation: low speed centrifuge, electrically heated dry bath pot, pipette, quantitative PCR instrument, etc.
      3) Add 10 microliters of proteinase K and 100 microliters of sample to be tested (EDTA anticoagulated whole blood) in a 1.5 milliliter centrifuge tube; mix, centrifuge at 2000 rpm for 10 seconds; add 200 microliters of buffer solution B; mix by inversion; set at 56° C. for 10 minutes, during which mix by inversion 2-3 times; add 200 microliters of anhydrous ethanol; mix by inversion; pour into an adsorption column; centrifuge at 12000 rpm 30 seconds; discard waste; place the adsorption column back into a collection tube; add 500 microliters of buffer C to the adsorption column; centrifuge at 1200 rpm 30 seconds; discard waste; place the adsorption column back into the collection tube; add 700 microliters of rinse solution W2 to the adsorption column; centrifuge at 12000 rpm 30 seconds; discard waste; place the adsorption column back into the collection tube; add 500 microliters of rinse solution W2 into the adsorption column; centrifuge at 12000 rpm for 30 seconds; discard waste; place the adsorption column back into the collection tube; centrifuged at 12000 rpm for 2 minutes; place the adsorption column in a new 1.5 milliliter centrifuge tube; set at room temperature for a few minutes to completely dry the residual rinse solution; add 100 microliters of elution buffer TE drop wise to the middle of the adsorption film; set at room temperature for 2-5 minutes; centrifuge at 12000 rpm for 2 minutes; and collect the genomic DNA in the collection tube.
   b. Remove from the refrigerator a fluorescent tube for personal use. Add probes, primers, paraffin oil, into the PCR reaction solution according to specified ratio. An operator adds sequentially. 3.7 microliters of deionized triple-distilled water, 10 microliters of MIX reaction solution, and 4.5 microliters of DNA extracted from the previous step. Carry out amplification on an instrument according to the following procedure: 95° C. 10 minutes—40 cycles (95° C. 10 seconds −60° C. 30 seconds).
   c. Finally, according to the amplification curve, determine the genotype of the tested sample. Administer drugs in accordance with the corresponding treatment plan.

What is claimed is:

1. A rs6311 detection kit, said detection kit comprises:
a probe;
a primer; and
a polymerase chain reaction solution.

2. The detection kit in claim 1, wherein said probe has a sequence as follows:
(SEQ. ID. NO. 3)
rs6311T-fam: CTGTGAGTGTCTGGC
(SEQ. ID. NO. 4)
rs6311C-vic: CTGTGAGTGTCCGGC

3. The detection kit in claim 1, wherein said primer has a sequence as follows:
(SEQ. ID. NO. 5)
rs6311-F: AGAGAGAACATAAATAAGGCTAGAAAACAGTA
(SEQ. ID. NO. 6)
rs6311-R: CACTGTTGGCTTTGGATGGA

4. The detection kit in claim 1, further comprising a fluorescent reaction rube wherein said probe and said primer are packed in said fluorescent reaction tube.

5. The detection kit in claim 1, further comprising triple-distilled deionized water.

6. The detection kit in claim 2, wherein concentration of said probe is at least 25 micro-molars in said triple-distilled deionized water.

7. The detection kit in claim 3, wherein concentration of said primer is at least 20 micro-molars in said triple distilled deionized water.

8. The detection kit in claim 1, wherein said detection kit comprises a first vial, containing
at least 0.1 microliter of said probe;
at least 0.8 microliter of said primer; and
said triple-distilled deionized water.

9. The detection kit in claim 1, wherein said detection kit comprises a second vial, comprising said polymerase chain reaction solution.

10. The detection kit in claim 1, further comprising paraffin oil.

11. The detection kit in claim 1, wherein said detection kit is stored at a temperature at least 20 degree centigrade below zero.

12. A method of using a rs6311 detection kit, comprising the steps of:
obtaining a whole blood sample from a patient;
extracting genomic DNA from said whole blood;
amplifying said genomic DNA via a polymerase chain reaction; and
determining genotype of said whole blood sample.

13. The method in claim 12, wherein said genomic DNA extracting step further comprising:
mixing said whole blood sample with a proteinase, a first buffer solution, and
anhydrous ethanol;
centrifuging;
setting at a temperature above room temperature;
adding a second buffer solution;
centrifuging;
adding a rinse solution;
centrifuging;
adding said rinse solution;
centrifuging;
setting at room temperature;
adding an elution buffer;
setting at room temperature;
centrifuging; and
collecting genomic DNA.

14. The method in claim 12, wherein said genomic DNA amplifying step further comprising:
obtaining said rs6311 detection kit;
adding triple-distilled deionized water;
adding a polymerase chain reaction solution;
adding said genomic DNA;
adding paraffin; and
amplifying said genomic DNA in a polymerase chain reaction instrument.

15. The method in claim 12, wherein said whole blood sample comprises an anti-conjugation agent.

16. The method in claim 12, wherein said detection kit comprises a probe, a primer, paraffin oil according to a pre-specified ratio.

17. The method in claim 12, wherein said genotype determining step further comprises performing single strand polymorphism analysis to determine genotype in said genomic DNA.

18. The method in claim 14, wherein said polymerase chain reaction is performed at a temperature cycle from 95 degrees to 60 degrees centigrade.
